(19) United States
(12) Patent Application Publication
(10) Pub. No.: US 2026/0020770 A1
(43) Pub. Date: Jan. 22, 2026

(54) BLOOD PRESSURE PULSE WAVEFORM ANALYSIS (PWA) TO ASSESS RISK OF PREECLAMPSIA

(71) Applicant: Georgia Tech Research Corporation, Atlanta, GA (US)

(72) Inventors: Rudolph Lawson Gleason, JR., Atlanta, GA (US); Julia Lins Arrighi Woodall, Atlanta, GA (US); Mahlet Yigeremu Gebremariam, Addis Ababa (ET)

(21) Appl. No.: 19/273,923

(22) Filed: Jul. 18, 2025

Related U.S. Application Data
(60) Provisional application No. 63/673,121, filed on Jul. 18, 2024.

(51) Int. Cl. A61B 5/021; A61B 5/00

(52) U.S. Cl. CPC A61B 5/02108; A61B 5/4343; A61B 5/7275

(57) ABSTRACT

A method for assessing the risk of preeclampsia in a pregnant subject is provided. The method can comprise: obtaining at least one blood pressure pulse waveform from at least one arterial site of the subject; determining a risk of preeclampsia in the subject, based at least in part on the at least one blood pressure pulse waveform; and generating an output indicative of the determined risk.

### CROSS-REFERENCE TO RELATED APPLICATIONS

This application claims the benefit of U.S. Provisional Application Ser. No. 63/673,121, filed on 18 Jul. 2024, which is incorporated herein by reference in its entirety as if fully set forth below.

### GOVERNMENT LICENSE RIGHTS

This invention was made with government support under 1R01HD100635 awarded by National Institutes of Health. The government has certain rights in this invention.

### FIELD OF THE DISCLOSURE

The various embodiments of the present disclosure relate generally to systems and method for assessing risk of preeclampsia in a patient using blood pressure pulse waveforms.

### BACKGROUND

Preeclampsia is a complex and potentially life-threatening condition that affects pregnant women worldwide, characterized by the onset of hypertension and proteinuria after 20 weeks of gestation. It is the second leading cause of maternal mortality globally, with a disproportionate impact on low- to middle-income countries (LMICs) such as Ethiopia, where access to healthcare resources is limited. The condition not only poses significant risks to maternal health but also affects fetal development, often leading to complications such as fetal growth restriction.

Current risk detection methods for preeclampsia primarily rely on high-resource technologies like Doppler ultrasound, which measures uterine artery pulsatility index (UA-PI) as a key indicator of risk. UA-PI is a shape characteristic of the uterine artery blood flow velocity waveform, and its elevation is associated with increased risk of preeclampsia. However, these technologies are costly and require specialized training, making them inaccessible in many LMICs. In developed countries, the Fetal Medicine Foundation's competing-risks model, which incorporates UA-PI along with maternal factors and biochemical markers, has shown efficacy in predicting preeclampsia risk. Yet, even this model faces limitations in LMICs due to the need for sophisticated equipment and trained personnel.

Given these challenges, there is a pressing need for alternative, low-cost screening methods that can be implemented in resource-limited settings.

### BRIEF SUMMARY

According to a first aspect of the present disclosure, a method for assessing the risk of preeclampsia in a pregnant subject is provided. The method can comprise: obtaining at least one blood pressure pulse waveform from at least one arterial site of the subject; determining a risk of preeclampsia in the subject, based at least in part on the at least one blood pressure pulse waveform; and generating an output indicative of the determined risk.

In any of the embodiments disclosed herein, determining the risk of preeclampsia can comprise predicting a uterine artery pulsatility index (UA-PI) based, at least in part, on the blood pressure pulse waveform.

In any of the embodiments disclosed herein, determining the risk of preeclampsia can further comprise comparing the predicted UA-PI to a threshold value.

In any of the embodiments disclosed herein, determining the risk of preeclampsia can comprise analyzing the obtained blood pressure pulse waveforms to identify shape features indicative of uterine artery blood flow velocity waveforms.

In any of the embodiments disclosed herein, the shape features can comprise diastolic notch depth, relative values of early or late diastolic pressures compared to a peak systolic pressure, local maxima or minima of the waveforms or derivatives thereof, principal companies identified with PCA, and/or waveform duration.

In any of the embodiments disclosed herein, determining a risk of preeclampsia in the subject can be further based, at least in part, on one or more of a maternal age of the subject, a current gestational timeline of the subject, a normal blood pressure of the subject, any prior preeclampsia diagnoses or other gestational diagnoses, or biomarkers measured from blood or urine in the subject.

In any of the embodiments disclosed herein, the arterial site can be selected from the group consisting of the brachial artery, femoral artery, radial artery, tibial artery, finger, and toc.

In any of the embodiments disclosed herein, determining a risk of preeclampsia in the subject can comprise performing waveform shape analysis (e.g., generalized transfer functions, Fourier transform functions, principal component analysis, PCA)) on the at least one blood pressure pulse waveform.

In any of the embodiments disclosed herein, the method can further comprise providing a recommendation for administration of a pharmaceutical agent if the determined risk in the output is above a predetermined threshold.

In any of the embodiments disclosed herein, obtaining at least one blood pressure pulse waveform from at least one arterial site of the subject can comprise obtaining a first blood pressure pulse waveform from the at least one arterial site of the subject at a first time during a gestational period of the subject and obtaining a second blood pressure pulse waveform from the at least one arterial site of the subject at a second time during a gestational period of the subject.

Accordingly to a second aspect of the present disclosure, a system for assessing risk of preeclampsia in a pregnant subject is provided. The system can comprise a processor and memory. The memory can comprise instructions that, when executed by the processor, cause the processor to: obtain at least one blood pressure pulse waveform from at least one arterial site of the subject; determine a risk of preeclampsia in the subject, based at least in part on the at least one blood pressure pulse waveform; and generate an output indicative of the determined risk.

In any of the embodiments disclosed herein, the system can further comprise a blood pressure pulse waveform measuring device configured to generate the at least one blood pressure pulse waveform.

In any of the embodiments disclosed herein, the blood pressure pulse waveform measuring device is selected from the group consisting of: oscillometric devices, applanation tonometry devices, volume clamp devices, photoplethysmography-based devices, Doppler ultrasound systems, and invasive arterial catheters.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIGS. 1A-E provide plots of hemodynamic markers for different forms of preeclampsia (PE), in accordance with some embodiments of the present disclosure, in which the individual data points are taken from literature reports for uncomplicated pregnancies, late forms of PE, and early forms of PE, and the lines indicate illustrative simulation results for uncomplicated, late PE, and early PE, in accordance with some embodiments of the present disclosure.

FIG. 2A provides blood pressure waveforms along the vascular tree, in accordance with some embodiments of the present disclosure.

FIG. 2B provides a plot showing that carotid-femoral pulse wave velocity (cf-PWV) decreases in the first half of uncomplicated (normal) pregnancy and increases in the second half, while in early and late forms of PE, cf-PWV is elevated by ˜1.13-1.18 fold early in pregnancy, then increases more dramatically as blood pressure increases.

FIG. 2C provides an illustration of a calculation of the augmentation index (AIₓ) of a pressure waveform, in accordance with some embodiments of the present disclosure.

FIG. 2D provides a plot showing that AIₓ-75 (i.e., AIₓ normalized to a heart rate of 75 bpm) in accordance with some embodiments of the present disclosure, because in normal pregnancy, AIₓ-75 decreases in the first trimester, then increases or remains nearly constant in the second and third trimesters, but with early and late forms of PE, AIₓ-75 is elevated, compared to normal pregnancy, particularly in the third trimester, when blood pressure is elevated.

FIGS. 3A-D provide a statistical shape modeling (SSM) analysis pipeline, in which PCA is performed on original waveforms from thousands or tens-of-thousands of measurements across subjects to identify the mean waveform and principal components, such that the 2p dimensional waveform

$${W}_{i}^{d}$$

is reduced to a m dimensional waveform

$${W}_{i}^{m} ,$$

in accordance with some embodiments of the present disclosure.

FIGS. 4A-D provide representative model outputs of blood flow waveforms, blood flow centerline velocity waveforms, blood pressure waveforms, and diameter waveforms, respectively, and how these waveforms evolve across gestation, in which the legend in FIG. 4A also described the curves in FIGS. 4B-D, in accordance with some embodiments of the present disclosure.

FIGS. 5A-C provide graphs showing shape changes in the uterine artery velocity and femoral and brachial artery pressure waveforms, respectively, from 12 to 20 weeks of gestation for a representative subject, in which the UA velocity was normalized by diving each waveform by the peak systolic velocity and the pressure was normalized so that the end diastolic pressure is 0 and the peak systolic pressure was 1, in accordance with some embodiments of the present disclosure.

FIGS. 5D-E provide graphs showing automatically-detected user-defined features of these curves shown in FIGS. 5A-C, in accordance with some embodiments of the present disclosure.

FIG. 5F provides a table showing mean, standard deviation, and p-values for select user-defined features from low (<1.5) versus high (>1.5) uterine artery pulsatility index (PI) values.

FIGS. 6A-D provide a modeling approach to predict uterine artery waveforms and other screening algorithm risk scores from measured blood pressure waveforms, in accordance with some embodiments of the present disclosure.

### DETAILED DESCRIPTION

Although preferred exemplary embodiments of the disclosure are explained in detail, it is to be understood that other exemplary embodiments are contemplated. Accordingly, it is not intended that the disclosure is limited in its scope to the details of construction and arrangement of components set forth in the following description or illustrated in the drawings. The disclosure is capable of other exemplary embodiments and of being practiced or carried out in various ways. Also, in describing the preferred exemplary embodiments, specific terminology will be resorted to for the sake of clarity.

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

As used in the specification and the appended claims, the singular forms “a,” “an” and “the” include plural referents unless the context clearly dictates otherwise.

Also, in describing the preferred exemplary embodiments, terminology will be resorted to for the sake of clarity. It is intended that each term contemplates its broadest meaning as understood by those skilled in the art and includes all technical equivalents which operate in a similar manner to accomplish a similar purpose.

Ranges can be expressed herein as from “about” or “approximately” one particular value and/or to “about” or “approximately” another particular value. When such a range is expressed, another exemplary embodiment includes from the one particular value and/or to the other particular value.

Similarly, as used herein, “substantially free” of something, or “substantially pure”, and like characterizations, can include both being “at least substantially free” of something, or “at least substantially pure”, and being “completely free” of something, or “completely pure”.

By “comprising” or “containing” or “including” is meant that at least the named compound, member, particle, or method step is present in the composition or article or method, but does not exclude the presence of other compounds, materials, particles, method steps, even if the other such compounds, material, particles, method steps have the same function as what is named.

Mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

The materials described as making up the various members of the invention are intended to be illustrative and not restrictive. Many suitable materials that would perform the same or a similar function as the materials described herein are intended to be embraced within the scope of the invention. Such other materials not described herein can include, but are not limited to, for example, materials that are developed after the time of the development of the invention.

Reference will now be made in detail to exemplary embodiments of the disclosed technology, examples of which are illustrated in the accompanying drawings and disclosed herein. Wherever convenient, the same references numbers will be used throughout the drawings to refer to the same or like parts.

Embodiments of the present disclosure provide systems and methods for assessing the risk of preeclampsia using blood pressure pulse waveforms. The method can involve collecting blood pressure pulse waveforms from various arterial sites, such as the femoral and brachial arteries, and analyzing these waveforms to predict uterine artery blood flow velocity waveforms and preeclampsia risk. The methods can leverage the correlation between easily acquired blood pressure pulse waveforms and uterine artery velocity waveforms, which are currently used to assess preeclampsia risk. By identifying novel shape features of these waveforms that correlate with uterine artery pulsatility index (UA-PI), embodiments of the present disclosure offer a more accurate and equitable strategy for preeclampsia risk assessment, potentially reducing reliance on costly and less accessible ultrasound technology.

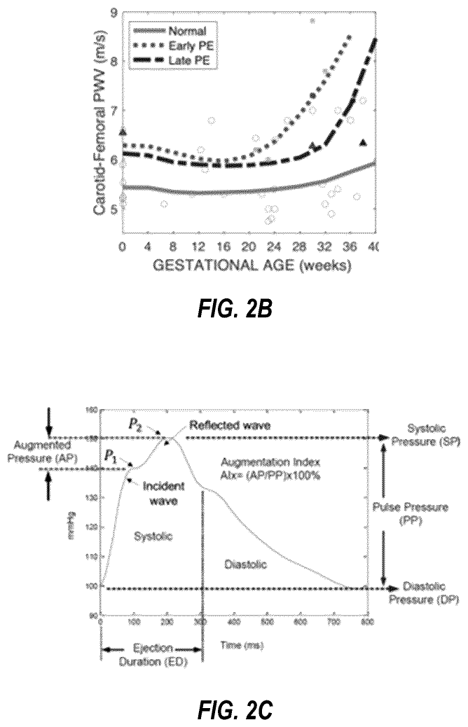

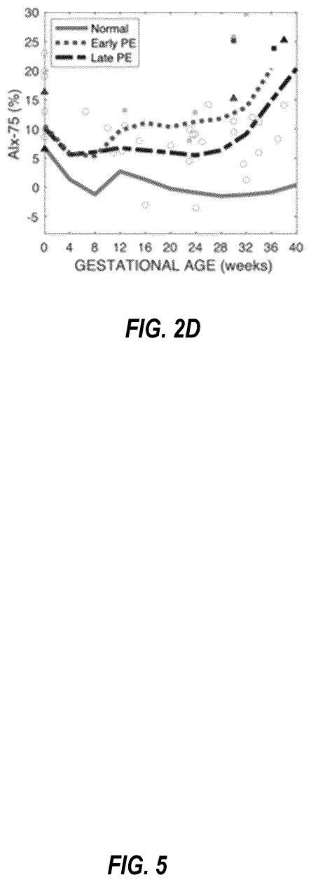

By way of background, each cardiac contraction sends a pressure pulse wave down the vascular tree (See FIGS. 2A-D). The speed and shape of this pulse wave change as it travels and the pulse wave speed and shape are governed by several features of the cardiovascular system, including heart rate, blood pressure, the stiffness of the arterial tree, and peripheral resistance and compliance of the distal vasculature. BP pulse waveforms are accessible at multiple locations in the vascular tree (e.g., common carotid, brachial (upper arm), radial (wrist), femoral (thigh), and tibial (ankle), arteries and the finger and toe) and can provide important information about vascular hemodynamics.

Pulse wave velocity (PWV) is typically determined by measuring the time it takes for the pressure pulse to travel from the heart to a specific location in the vasculature (See FIG. 2A) and the distance between the heart and the measurement location. Transit time divided by distance traveled yields the PWV. Carotid-femoral PWV (cf-PWV) is considered the gold-standard, clinically tractable, surrogate marker for arterial stiffness. Aortic stiffness and cf-PWV increase with age and cf-PWV is considered a composite metric of vascular age; cf-PWV is a key, independent risk factor for future cardiovascular events. In normal pregnancy, the cf-PWV decreases in the first half of pregnancy and increase in the second half (See FIG. 2B). In early and late forms of PE, PWV is elevated by ˜1.13-1.18 fold early in pregnancy, then increases more dramatically as BP increases.

Pulse Wave Analysis (PWA) uses measured BP waveforms from one location (or multiple locations) to estimate BP waveforms or other hemodynamic metrics at another location. One common application of PWA is using the brachial or radial artery BP waveform to estimate the BP waveform in the ascending aorta, which is then used to estimate left ventricular load, arterial stiffness (via the augmentation index, AIₓ), CO, stroke volume, and total peripheral resistance (TPR). The aortic BP waveform has two characteristic pressures, P₁ and P₂ (See FIG. 2C), which represent the systolic peak of the advancing BP waveform (P₁) and the peak of the composite of the advancing and reflecting waveforms (P₂). AIₓ is the ratio of the augmentation pressure (AP) to the pulse pressure (PP). It has been shown that AIₓ is correlated with heart rate and defined

$${AI}_{x}^{75} = 0.39 ( H R - 75 ) + {AI}_{x} .$$

In normal pregnancy,

$${AI}_{x}^{75}$$

decreases in the first trimester, then increases or remains nearly constant in the second and third trimester (See FIG. 2D). With early and late forms of preeclampsia,

$${AI}_{x}^{75}$$

is elevated, compared to normal pregnancy, particularly in the third trimester, when BP is elevated. While PWA metrics have been shown to correlate well with UtA-PI and PE, these correlations are modest and PWA has not been exploited to assess PE risk.

Accordingly, the present disclosure utilizes novel BP pulse wave characteristics that correlate much more strongly to UtA-PI, compared to cf-PWV and

$${AI}_{x}^{75} .$$

In particular, some embodiments of the present disclosure exploit this observation and use novel PWA methods, including statistical shape modeling combined with machine learning, and physics-informed 1D fluid-solid-growth-remodeling (FSGR) modeling to predict the shape of the UtA blood flow velocity waveforms and PE risk, based on BP waveforms.

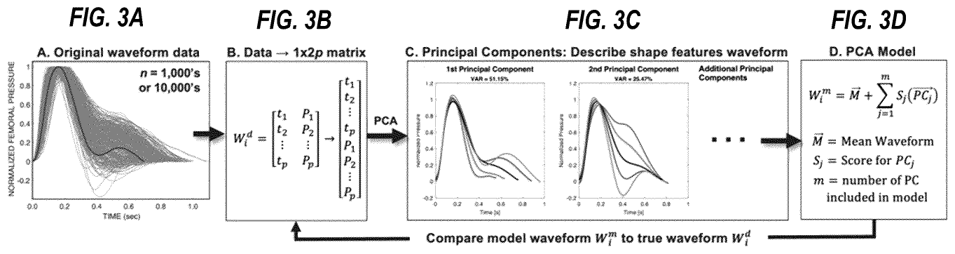

Embodiments of the present disclosure can assess risk of PE based on blood pressure pulse waveforms. This can be done, in part, by characterizing shape of the pressure and blood flow velocity waveform data; e.g., a statistical shape modeling (SSM), generalized transfer functions, Fourier transfer functions, user-defined waveform features, and the like, to extract curve shape features from pressure or velocity versus time curves (See FIGS. 3A-D). Each original waveforms (e.g., BP or flow velocity waveforms) across 1,000's or 10,000's of measurements (See FIG. 3A) can be organized into a structured matrix of dimension 1-by-2p (See FIG. 3B), where p is the number of data points within each waveform. Each waveform can be combined into a single matrix of dimension n-by-2p, where n is the number of individual waveforms being included in the analysis. PCA can be performed on the n-by-2p matrix to yield a mean waveform \vec{M} (black curves in FIG. 3C) and principal components that describe the characteristic shape features that differ between different waveforms. A model waveform

$$( {W}_{i}^{m} ) ,$$

can be generated for each original waveform

$$( {W}_{i}^{d} ) ,$$

which can combine the mean waveform \vec{M} and unique scores (Sⱼ) for each principal component (\vec{PCⱼ}) included in the model (See FIG. 3D); m represents the total number of principal components included in the PCA model. The accuracy of the model can be assessed by comparing

$${W}_{i}^{m}$$

with

$${W}_{i}^{d}$$

and model accuracy can be improved by adding more principal components (i.e., increasing m). In this way, the 2p dimensional waveform

$${W}_{i}^{d}$$

can be reduced to a m dimensional waveform

$${W}_{i}^{m} .$$

The m principal component scores Sⱼ(\vec{PCⱼ}) can then be compared across groups and used as inputs for machine learning algorithms to relate the shape of BP and velocity waveforms at site with those at different measurement sites in the same subject; e.g. using femoral artery pressure Sⱼ(\vec{PCⱼ}) to predict uterine artery flow Sⱼ(\vec{PCⱼ}) and thus, uterine artery

$${W}_{i}^{m} .$$

This can be compared to traditional curve fitting methods and ‘user-defined’ features. Systolic, diastolic, pulse, and augmentation pressures are examples of user-defined features of pressure waveforms. UtA-PI, UtA resistivity index (UtA-RI), and UtA systolic-to-diastolic ratio (UtA-S/D) are user-defined features of the uterine artery blood flow velocity waveform.

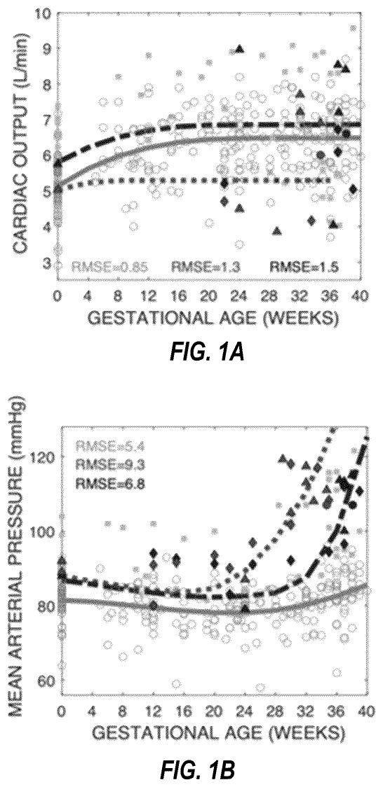

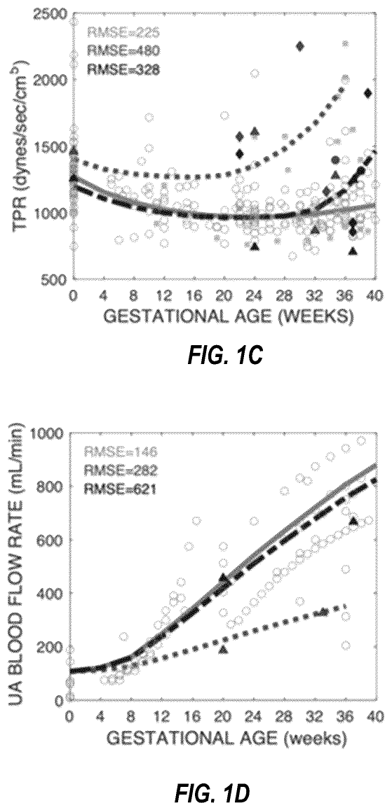

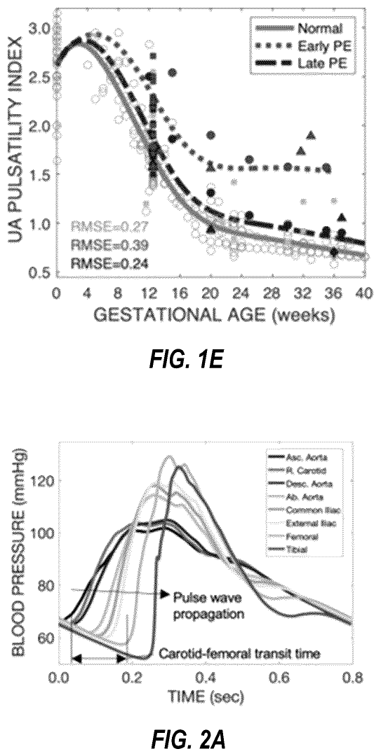

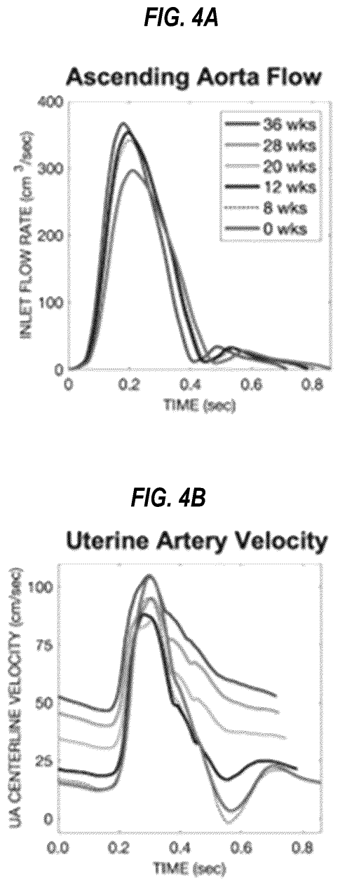

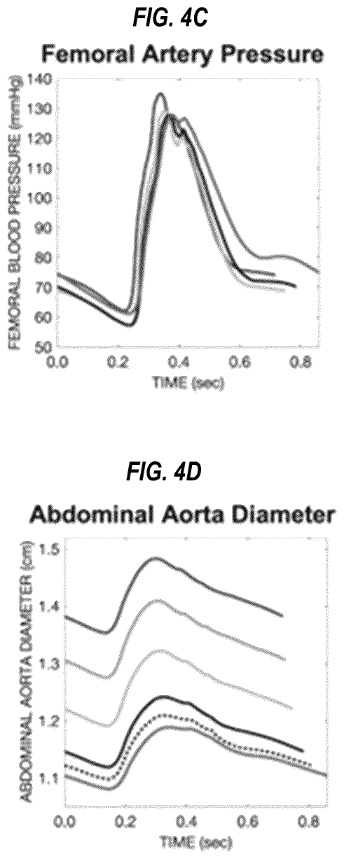

A novel computational modeling framework was developed to describe the changes in maternal hemodynamics and vascular growth and remodeling across gestation in normal pregnancies and pregnancies complicated with early and late forms of preeclampsia. This started with a defined arterial network adding a model uterine vasculature. Arteries can be modeled as tapered, elastic tubes in a one-dimensional (1D) pulsatile fluid flow framework. The balance of mass and balance of linear momentum equations can be solved numerically, with models for the velocity profile and wall distensibility. The inlet boundary condition can be the blood flow rate waveform at the inlet of the ascending aorta (See FIG. 4A). The outlet boundary conditions can be zero-dimensional, three-element Windkessel models at each terminal artery, which describe the resistance and compliance of the vasculature distal to the termination site in terms of three parameters (R₁, R₂, and C_{T}). For each artery segment, the inlet and outlet diameter, artery length, and artery distensibility can be prescribed. One can start with published and validated model parameters then employ a growth and remodeling approach to adapt the maternal vasculature (i.e., adjust the “fluid-solid” model parameters) to matched literature values. After prescribing the inlet boundary condition to match literature values, the terminal resistances were adjusted (R₁, R₂) until known blood flow values were matched, as closely as possible. Next, the radii of the artery segments were adjusted to match wall shear stress values based on literature data. Finally, terminal compliances (C_{T}) and the distensibility of each artery segment were adjusted to match published shape features of pressure and velocity waveforms; e.g., SBP, DBP, and UtA-PI. Illustrative model outputs for blood flow waveforms, blood velocity waveforms, BP waveforms, and diameter waveforms are shown in FIGS. 4A-D. Illustrative results for the differences in the time-course of key hemodynamic parameters versus gestation for normal and early- and late-PE are shown in FIGS. 1A-E.

Some embodiments of the present disclosure can employ and significantly advance these models by collecting a large set of validation data, through echocardiography, Doppler, M-mode, and B-mode ultrasound for diameter and velocity waveforms at 16 distinct arterial sites and BP waveforms at 14 distinct sites. With these data, subject-specific 1D FSGR models can be generated for subjects with uncomplicated pregnancies and pregnancies with early onset, late preterm, and term PE. These models and data can (i) provide an accurate characterization the etiologies of PE and (ii) inform risk assessment models to improve model accuracy.

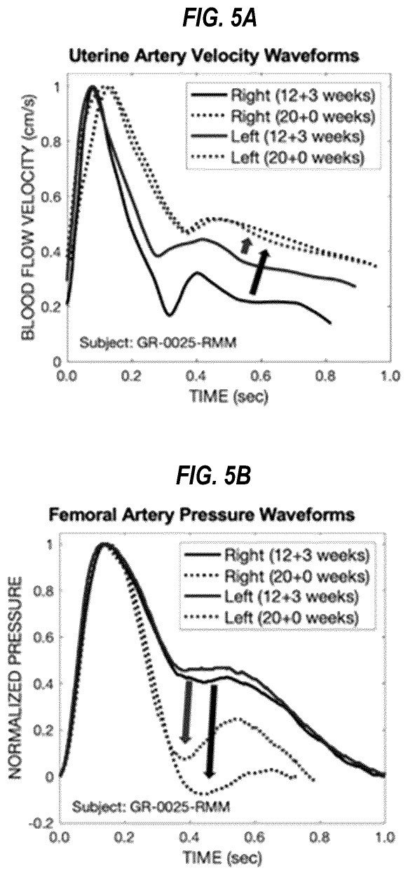

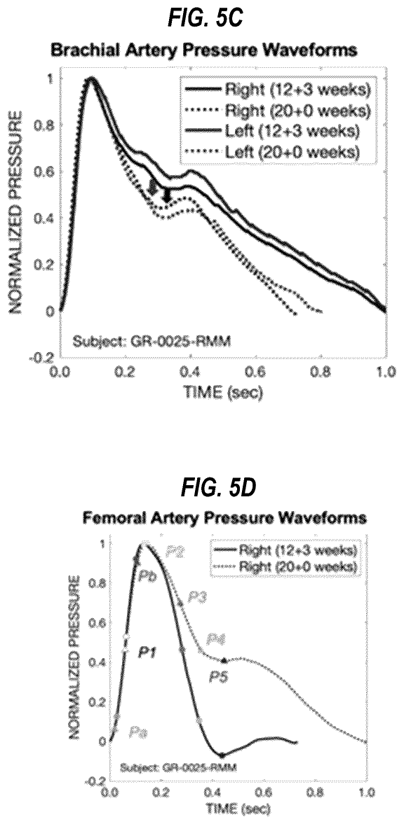

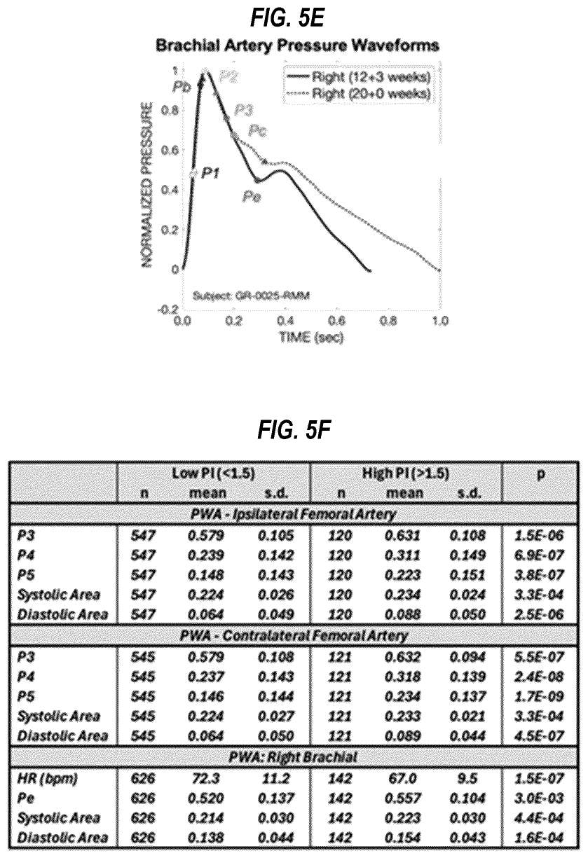

As discussed above, embodiments of the present disclosure can perform PWA to use blood pressure pulse waveforms to accurately predict uterine artery blood flow velocity waveforms. UtA-PI decreases dramatically from 8 to 20 weeks of gestation (See FIG. 1E), primarily due to increased uterine artery diastolic blood flow. Elevated UtA-PI at 11-14 weeks of gestation is a key indicator PE risk and indicates that the perfusion of the uterine vasculature is not increasing as it should with gestation. From a physics standpoint, increases in perfusion of the uterine vasculature will affect the hemodynamics in other nearby (and distant) vascular beds. Data suggest that the shape of the BP waveforms at the femoral and brachial arteries correlate well with the shape of the UtA blood flow velocity waveforms. Thus, embodiments of the present disclosure can make use of the correlation between user-defined shape features of the femoral and brachial artery pressure waveforms and the UtA blood flow waveforms (see FIGS. 5A-F). Specifically, there is a significant decrease in the diastolic portion of the BP waveform in the femoral and brachial artery (see FIGS. 5B-C, see arrows) which is coincident with the increase in diastolic velocity of the UtA (see FIG. 5A). In this subject, there is even a greater decrease in early diastolic pressure on the right femoral artery where we also observed a greater increase in right UtA diastolic velocity. This observation is generally true across all analyzed subjects, as shown in FIGS. 5C-F. The user-defined features (e.g., FIGS. 5D-E) of the femoral and brachial artery BP waveforms can be significantly different (p values=10⁻³ to 10⁻⁹) between subjects with high versus low UtA-PI values, as shown in FIG. 5F. These observations indicate that BP waveforms, measured at the right and left carotid, brachial, femoral, radial, tibial arteries, index fingers, and/or toes can predict UtA-PI within 10% for >90% of the subject measurement taken at the first antenatal care visit.

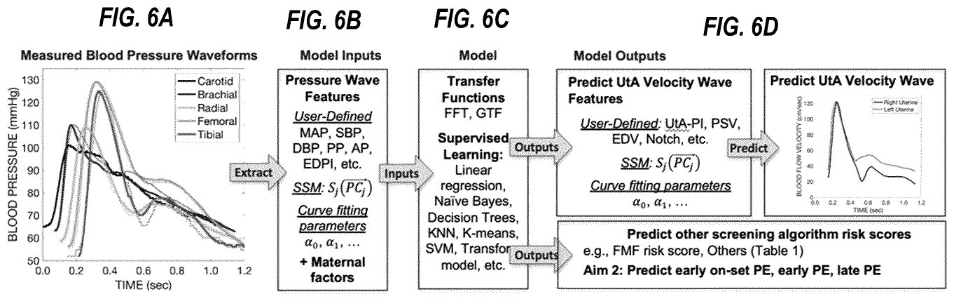

Following our data analysis pipeline (see FIGS. 6A-D) all BP waveforms collected during the 1^{st} trimester visit can be used as predictors of UtA blood flow velocity waveform features (see FIG. 6A). Transfer functions (e.g., Fast Fourier Transform, FFT, Generalized Transfer Function, GTF) were developed to generate models that directly “transform” the set of BP waveforms to predict the set of UtA velocity waveforms. Alternatively, BP waveform features can be extracted from the waveforms. Features may arise from user-defined measurements, SSM scores, and traditional curve fitting parameters (see FIG. 6B). User-defined measurements include well-known features, such as MAP, SBP, DBP, PP, and AP, and novel features, such as P3, P4, and P5 (see FIGS. 5A-F). SSM scores for the pressure waveforms can be determined using the PCA pipeline (see FIGS. 3A-D) described above. SSM scores can be considered as model inputs. Traditional curve fitting parameters can also be determined. Multiple models can be used, including Fourier series models

$$( y = {a}_{0} + {\sum }_{i = 1}^{n} {a}_{i} cos ( i w x ) + {b}_{i} sin ( i w x ) )$$

and polynomial

$$( y = {b}_{0} + {\sum }_{i = 1}^{n} {b}_{i} {x}^{i} )$$

models, and the fitted parameters can be used as candidate model inputs. With this long list of potential model inputs, some embodiments of the present disclosure can employ supervised machine learning strategies, such as linear/logistical regression, Naïve Bayes, decision tree, random forest, K-nearest neighbor (KNN), K-means, support vector machine (SVM) algorithms, and Transformer models amongst others (see FIG. 6C). These machine learning models can be trained by comparing predicted uterine artery velocity waveform features to Doppler ultrasound measured values of those features (see FIG. 6D). The models can also predict other screening algorithm scores (e.g., the FMF algorithm) from pressure wave features and other ‘low resource’ features such as maternal factors.

As discussed above, embodiments of the present disclosure are generally directed to systems and methods for assessing the risk of preeclampsia in a pregnant subject. At a high level, the method can comprise: obtaining at least one blood pressure pulse waveform from at least one arterial site of the subject; determining a risk of preeclampsia in the subject, based at least in part on the at least one blood pressure pulse waveform; and generating an output indicative of the determined risk. In come embodiments of the present disclosure, the method can be formed, entirely, or partly by a computer. For example, a system for performing the methods disclosed herein can comprise one or more processors and one or more memories. The one or more memories can comprise instructions that, when executed by the one or more processors, cause the one or more processors to perform one or more of the various method steps disclosed herein. Additionally, in some embodiments one or more machine learning models can be utilized to determine the risk of PE based on the blood pressure pulse waveforms.

Obtaining blood pressure pulse waveforms can be performed many different ways. In some embodiments, blood pressure pulse waveforms can be taken at a remote location and transmitted to remote computing system for processing. In some embodiments, obtaining the blood pressure pulse waveforms can include measuring blood pressure pulses using many different devices known in the art, including, but not limited to, oscillometric devices (e.g., Vicorder, Mobil-O-Graph), applanation tonometry devices (e.g., SphygmoCor, Millar tonometers), volume clamp devices (e.g., Finapres, CNAP monitor), photoplethysmography-based devices (e.g., wearable pulse oximeters, Empatica E4), Doppler ultrasound systems (e.g., GE Vivid, Philips Affiniti), and invasive arterial catheters (e.g., fluid-filled or pressure-tip catheters from Millar). and the like.

The blood pressure pulse waveforms can be obtained from many different locations on the subject, including, but not limited to, brachial artery, femoral artery, radial artery, tibial artery, and the like. In some embodiments, multiple blood pressure pulse waveforms can be collected from multiple locations on a single subject. In some embodiments, only a single blood pressure pulse waveform is collected for a single subject.

Additionally, in some embodiments, blood pressure pulse waveforms can be obtained over varying time periods, e.g., a first pulse waveform at a first time during the gestational period and a second pulse waveform at a second time during the gestational period of the subject. By obtaining pulse waveforms at different times, risk of PE can be evaluated at different points during the gestational period.

As discussed above, there is a correlation between blood pressure pulse waveforms, uterine artery blood flow velocity waveforms, and uterine artery pulsatility index (UA-PI). Thus, in some embodiments, principal component analysis (PCA) can be performed on the obtained blood pressure pulse waveform. For example, blood pressure pulse waveforms can be analyzed to identify shape features indicative of uterine artery blood flow velocity waveforms. In some embodiments, the analysis These shape features can include, but are not limited to, diastolic notch depth and waveform duration. Based on these identified shape features, UA-PI can be predicted, and the risk of PE can be accessed based on the predicted UA-PI. For example, the predicted UA-PI can be compared to a threshold value to make a determination as to a risk of PE.

As those skilled in the art would understand, to determine the risk of PE, other maternal factors/parameters, in addition to the blood pressure pulse waveforms, can also be considered, these factors can include, but are not limited to, a maternal age of the subject, a current gestational timeline of the subject, a normal blood pressure of the subject, and any prior preeclampsia diagnoses in the subject.

After the risk of PE is determined, in some embodiments, an output is generated indicative of the determined risk of PE. The output can be many different outputs indicative of the risk. In some embodiments, the risk can be output as a analog score (e.g., higher value equates to higher risk). In some embodiments, the output can be a binary output in which a first value is indicative of high risk of PE and a second value is indicative of low risk of PE. In some embodiments, the output can be provided immediately by the same device used to measure blood pressure pulse waveforms (e.g., an indicator light for a binary output or a digital number for an analog signal).

Additionally, in some embodiments, a recommendation can be made for administering a pharmaceutical agent if the determined risk is above a threshold. For example, a recommendation can be provided to the subject to take an aspirin. Additionally, in some embodiments, a recommended dosing regimen for the pharmaceutical agent can also be provided.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

What is claimed is:

1. A method for assessing the risk of preeclampsia in a pregnant subject, comprising:
obtaining at least one blood pressure pulse waveform from at least one arterial site of the subject;
determining a risk of preeclampsia in the subject, based at least in part on the at least one blood pressure pulse waveform; and
generating an output indicative of the determined risk.

2. The method of claim 1, wherein determining the risk of preeclampsia comprises predicting a uterine artery pulsatility index (UA-PI) based, at least in part, on the blood pressure pulse waveform.

3. The method of claim 2, wherein determining the risk of preeclampsia further comprises comparing the predicted UA-PI to a threshold value.

4. The method of claim 1, wherein determining the risk of preeclampsia comprises analyzing the obtained blood pressure pulse waveforms to identify shape features indicative of uterine artery blood flow velocity waveforms.

5. The method of claim 4, wherein the shape features comprise diastolic notch depth and waveform duration.

6. The method of claim 1, wherein determining a risk of preeclampsia in the subject is further based, at least in part on, one or more or a maternal age of the subject, a current gestational timeline of the subject, a normal blood pressure of the subject, and any prior preeclampsia diagnoses in the subject.

7. The method of claim 1, wherein the arterial site is selected from the group consisting of the brachial artery, femoral artery, radial artery, and tibial artery.

8. The method of claim 1, wherein determining a risk of preeclampsia in the subject comprises performing principal component analysis (PCA) on the at least one blood pressure pulse waveform.

9. The method of claim 1, further comprising providing a recommendation for administration of a pharmaceutical agent if the based on the determined risk in the output is above a predetermined threshold.

10. The method of claim 1, wherein obtaining at least one blood pressure pulse waveform from at least one arterial site of the subject comprises obtaining a first blood pressure pulse waveform from the at least one arterial site of the subject at a first time during a gestational period of the subject and obtaining a second blood pressure pulse waveform from the at least one arterial site of the subject at a first time during a gestational period of the subject.

11. A system for assessing risk of preeclampsia in a pregnant subject, comprising a processor and memory, the memory comprising instructions that, when executed by the processor, cause the processor to:
obtain at least one blood pressure pulse waveform from at least one arterial site of the subject;
determine a risk of preeclampsia in the subject, based at least in part on the at least one blood pressure pulse waveform; and
generate an output indicative of the determined risk.

12. The system of claim 11, further comprising a blood pressure pulse waveform measuring device configured to generate the at least one blood pressure pulse waveform.

13. The system of claim 11, wherein the blood pressure pulse waveform measuring device is selected from the group consisting of oscillometric devices, applanation tonometry devices, volume clamp devices, photoplethysmography-based devices, Doppler ultrasound systems, and invasive arterial catheters.

14. The system of claim 11, wherein the instructions, when executed by the processor, further cause the processor to: determine the risk of preeclampsia at least in part by predicting a uterine artery pulsatility index (UA-PI) based, at least in part, on the blood pressure pulse waveform.

15. The system of claim 14, wherein the instructions, when executed by the processor, further cause the processor to determine the risk of preeclampsia at least in part by comparing the predicted UA-PI to a threshold value.

16. The system of claim 11, wherein the instructions, when executed by the processor, further cause the processor to determine the risk of preeclampsia at least in part by analyzing the obtained blood pressure pulse waveforms to identify shape features indicative of uterine artery blood flow velocity waveforms.

17. The system of claim 16, wherein the shape features comprise diastolic notch depth and waveform duration.

18. The system of claim 11, wherein the arterial site is selected from the group consisting of the brachial artery, femoral artery, radial artery, and tibial artery.

19. The system of claim 11, wherein the instructions, when executed by the processor, further cause the processor to determine a risk of preeclampsia in the subject at least in party by performing principal component analysis (PCA) on the at least one blood pressure pulse waveform.

20. The method of claim 11, wherein the instructions, when executed by the processor, further cause the processor to provide a recommendation for administration of a pharmaceutical agent if the based on the determined risk in the output is above a predetermined threshold.
